(19) United States
(12) Patent Application Publication
(10) Pub. No.: US 2026/0069629 A1
(43) Pub. Date: Mar. 12, 2026

(54) RECEPTACLE, DEVICE FOR RELEASING CARBON MONOXIDE, TREATMENT SYSTEM, SET FOR TREATING LIVING CELLS, METHOD OF TREATING A MAMMAL, AND CARBON MONOXIDE

(71) Applicant: Julius-Maximilians-Universität Würzburg, in Vertretung des Freistaates Bayern, Würzburg (DE)

(72) Inventors: Wolfgang Schmehl, Würzburg (DE); Lorenz Meinel, Würzburg (DE); Tessa Lühmann, Würzburg (DE); Simon Reiländer, Würzburg (DE); Christoph Kessler, Würzburg (DE); Marcus Gutmann, Würzburg (DE)

(73) Assignee: Julius-Maximilians-Universität Würzburg, in Vertretung des Freistaates Bayern, Würzburg (DE)

(21) Appl. No.: 19/108,129

(22) PCT Filed: Sep. 8, 2023
(86) PCT No.: PCT/EP2023/074808

§ 371 Date: Feb. 28, 2025

(30) Foreign Application Priority Data
Sep. 9, 2022 (EP) 22194868.0
Dec. 23, 2022 (EP) 22216317.2

(51) Int. Cl. A61K 33/00; A61K 9/48; C12M 1/42; C12N 5/076

(52) U.S. Cl. CPC A61K 33/00; A61K 9/4808; C12M 35/08; C12N 5/061; C12N 2501/999

(57) ABSTRACT

The present invention relates to a receptacle (10) configured to be inserted into a human body and/or an animal body, including a housing (12) and a chamber (14) defined within the housing (12). The chamber (14) is configured to receive a main substance (42) configured to selectively emit carbon monoxide after being activated by an activation substance (44). The chamber (14) is enclosed by a wall (16) of the housing (12) which is permeable to the carbon monoxide emitted by the main substance (42) such that the receptacle (10) can release the carbon monoxide emitted by the main substance (42) to an environment. The receptacle (10) is configured such that the activation substance (44) can be introduced into the chamber (14) from outside of the housing (12), in a state in which the main substance (42) is already arranged within the chamber (14). The present invention further relates to a device (40) for selectively releasing carbon monoxide, a treatment system (60; 80) and a set for treating biological cells, preferably living cells, a method for treating a mammal, a use of carbon monoxide, and method of treating biological cells.

A wide range of various substances, e.g., gases and/or liquids, are used for therapeutic purposes, e.g., to treat one or more diseases. The respective substances may be applied externally, e.g., onto the skin of a subject, e.g., a human and/or an animal. However, in some cases, it may be necessary and/or more effective to introduce the respective substance into the subject's body.

In some cases, it may be advantageous to store the respective substance in a housing, e.g., a capsule, to facilitate administering the respective substance to the respective treatment site, e.g., into the respective subject's body. This may allow the respective substance to be released at the treatment site, e.g., within the subject's body, in particular once the housing, e.g., capsule, has reached a particular region, e.g., a target region, in the subject's body. This may enhance the therapeutic effectiveness of the substance and/or prevent, or at least limit, adverse effects the substance may have on one or more regions of the subject's body.

Carbon monoxide (CO), for instance, is known to have advantageous therapeutic effects on humans and/or animals. For instance, in case one or more stress factors occur in a subject's body, the body may naturally initiate an increased production of carbon monoxide to trigger, or increase, one or more counter-mechanisms and/or defence mechanisms in response to the stress factors. Hence, supplementing carbon monoxide exogenously as a medical substance has a relatively high therapeutic potential.

However, there are several challenges to administering carbon monoxide to the respective subject. For instance, carbon monoxide is generally considered to be a toxic gas. In particular, carbon monoxide may mix and bind with hemoglobin in the blood of a subject, if the subject is exposed to carbon monoxide to a particular extent, e.g., if the carbon monoxide is inhaled. As a result, less oxygen may be transported to body tissues and vital organs, such as the brain and heart, which may have adverse effects, such as reversible and/or irreversible damage to a human body and/or animal body, even death.

Thus, a safe and/or controlled means of applying carbon monoxide is required. The prior art has proposed carbon monoxide releasing systems.

For instance, EP 3 242 552 B1, herein incorporated by reference in its entirety, discloses a gas releasing device which includes two compartments. One of the compartments contains carbon monoxide releasing molecules (CORM), whereas the other compartment contains an activation substance which triggers the release of carbon monoxide from the carbon monoxide releasing molecules. Once both substances from both compartments are combined, a chemical reaction which results in the release of carbon monoxide is initiated. Both compartments are surrounded by a gas permeable membrane which is permeable to carbon monoxide. The membrane is impermeable to the carbon monoxide releasing molecules, the activation substance and reaction by-products.

Moreover, WO 2021/074159 A1, herein incorporated by reference in its entirety, describes a membrane-based gas releasing device for oral application. The device includes two compartments which are separated by an inner septum and surrounded by a gas permeable membrane. One of the compartments is loaded with carbon monoxide releasing molecules, whereas the other compartment contains an activation substance which triggers the release of carbon monoxide from the carbon monoxide releasing molecules. One of the compartments includes an obliquely tapered hollow tip open end which can be shifted towards the inner septum, by squeezing the device, to break the inner septum to cause the carbon monoxide releasing molecules and the activation substance to mix, which results in carbon monoxide being released via the gas permeable membrane.

Furthermore, WO 2021/180908 A1, herein incorporated by reference in its entirety, discloses a therapeutic system configured for external treatment of wounds, inflammatory diseases of the skin, and inflammatory diseases of subcutaneous skin tissue, joints and tendons. The therapeutic system includes a reaction chamber comprising carbon monoxide releasing molecules and a gas-permeable and liquid- and solid-impermeable membrane. The carbon monoxide releasing molecules can be mixed with a carbon monoxide release triggering compound in the reaction chamber.

However, several drawbacks exist with respect to the carbon monoxide releasing devices known from the prior art. For instance, the configurations of the carbon monoxide releasing devices known from the prior art, e.g., the two-compartment construction of the device disclosed in WO 2021/074159 A1, are relatively complex which makes them relatively bulky and/or potentially prone to faults. Moreover, due to the relatively complex configurations, manufacturing the respective carbon monoxide releasing devices known from the prior art may also be relatively cost-intensive and/or relatively time-consuming and/or relatively prone to faults. Moreover, the system described in WO 2021/180908 A1 is specifically configured for treating an external surface of a subject, in particular the skin of the subject. For this purpose, the system described in WO 2021/180908 A1 includes a relatively complex mix of materials and a gas-impermeable portion to direct the released carbon monoxide towards the skin. Hence, the system lacks flexibility for a wider range of applications.

Various applications of carbon monoxide to different diseases and respective signalling mechanisms are discussed, e.g., in Gibbons et al., Carbon monoxide in gastrointestinal physiology and its potential in therapeutics, Aliment Pharmacol Ther. 2013 October; 38(7): 689-702.

Thus, the object of the present invention is to provide an improved means for administering carbon monoxide to a subject.

A first aspect of the present disclosure relates to a receptacle. The receptacle may be configured to be inserted into a human body and/or an animal body, preferably via one or more natural orifices of the respective body. The receptacle may include at least one housing and at least one chamber defined within the housing. The chamber may be configured to receive and store at least one main substance configured to selectively emit carbon monoxide. The main substance may be configured to emit carbon monoxide after being activated by at least one activation substance. The main substance may be configured to emit carbon monoxide substantially immediately after the main substance and the activation substance mix. Alternatively, the main substance may be configured to emit carbon monoxide in a time-delayer manner, i.e., after a certain time has lapsed after the main substance and the activation substance mix. The reaction between the main substance and the activation, which results in the main substance emitting carbon monoxide, may also be heat-dependent substance, e.g., such that the main substance begins emitting carbon monoxide when the mixture of the main substance and the activation substance is heated to a certain threshold temperature, e.g., by means of body heat within a human body and/or an animal body.

The chamber may be enclosed by at least one wall of the housing, wherein at least a section of the wall is at least partially permeable to the carbon monoxide emitted by the main substance such that the receptacle can at least partially release the carbon monoxide emitted by the main substance to an environment.

The “environment” may be a treatment site, e.g., within a human body and/or an animal body, or in a container for receiving biological cells, preferably living cells, e.g., immune cells, such as CAR T cells and/or stem cells, and/or gametes, such as sperm and/or oocytes, in which the cells are to be treated by the carbon monoxide released by the receptacle.

The receptacle may be configured such that the activation substance can be introduced into the chamber from outside of the housing, in a state in which the main substance is already arranged within the chamber. Once the activation substance has been introduced into the chamber, the activation substance and the main substance may react to release carbon monoxide via the wall of the housing.

Thus, in contrast to the respective carbon monoxide releasing devices known from the prior art, the activation substance may not be housed within the receptacle. Instead, the activation substance may be introduced into the chamber from outside of the housing, in a state in which the main substance is already arranged within the chamber, i.e., in a state in which the main substance is sealed within the chamber from the environment, preferably in a substantially fluid-tight and/or gas-tight manner. In other words, the main substance may be stored in the chamber until the receptacle is used. Before administering the receptacle, the activation substance may be introduced into the chamber from outside of the housing to activate the main substance. This may allow the construction of the receptacle to be simplified. In particular, since the activation substance can be introduced into the chamber from outside of the housing, in a state in which the main substance is already arranged within the chamber, the receptacle may not be required to include a separate chamber to house the activation substance prior to mixing of the main substance and the activation substance. Thus, the receptacle may be configured in a relatively simple manner, e.g., by including only a single chamber for housing the main substance, wherein the activation substance may be introduced directly into the chamber which houses the main substance such that the main substance and the activation can be mixed in the chamber. Moreover, one or more means for separating the activation substance and the main substance are not required and can therefore be omitted, in contrast to the carbon monoxide releasing devices known from the prior art, e.g., in EP 3 242 552 B1 and WO 2021/074159 A1. This may also provide a more compact receptacle compared with the carbon monoxide releasing devices known from the prior art which may facilitate the application of the receptacle (e.g., ingestion).

Furthermore, due to the simplified construction of the receptacle, as detailed above, manufacturing the receptacle may also be simplified, which may reduce the manufacturing efforts, e.g., the manufacturing costs and/or manufacturing time, of the receptacle. Moreover, the manufacturing process for manufacturing the receptacle may be generally less prone to faults.

In addition, configuring the receptacle such that the activation substance can be introduced into the chamber from outside of the housing, in a state in which the main substance is already arranged within the chamber, may provide a relatively simple and/or reliable activation process for activating the carbon monoxide releasing main substance. Thus, a relatively safe and/or relatively reliable mechanism for releasing carbon monoxide may be provided which may prevent, or at least reduce the risk of, adverse effects which the carbon monoxide releasing main substance and the activation substance may have on the subject which is being treated by means of the receptacle. For instance, carbon monoxide releasing substances and/or the respective activation substances may be toxic to the subject, if the subject is exposed to the carbon monoxide releasing substances and/or the respective activation substances, e.g., due to a fault in the construction of the receptacle and/or due to a faulty activation of the receptacle.

In addition, configuring the receptacle to be inserted into a human body and/or an animal body may increase the range of potential applications of the receptacle which may increase the flexibility of use of the receptacle. For instance, the receptacle may be configured for use in-vivo, e.g., by introducing the receptacle rectally and/or orally into a subject's body. For instance, the entire receptacle may be configured as a capsule, e.g., a capsule configured to be inserted, preferably manually, into one or more body cavities of an animal and/or a human, e.g., by oral administration and/or rectal administration to an animal and/or a human, e.g., a child and/or an adult, and/or into an ear of an animal and/or a human for treatment of, e.g., (but not limited to) otitis media and/or other inflammatory diseases where a targeted release of CO may be of therapeutic use, and/or into a vagina of an animal and/or a human.

Preferably, the receptacle is configured to be ingested, e.g., swallowed, by a human and/or an animal. Moreover, configuring the receptacle to be inserted into a human body and/or an animal body still allows the receptacle to be applied in a variety of further manners, e.g., by providing therapeutic treatment ex-vivo, such as by arranging the receptacle in a container which houses one or more biological cells, preferably one or more living cells, e.g., immune cells, such as CAR T cells and/or stem cells, and/or gametes, such as sperm or oocytes, to therapeutically treat and/or avoid degradation of the biological cells ex-vivo. Preferably, the receptacle is configured to be inserted into a human body and/or an animal body via one or more natural orifices of the respective body.

By contrast, the system described in WO 2021/180908 A1 is specifically configured for treating an external surface of a subject, in particular a skin of the subject. For this purpose, the system described in WO 2021/180908 A1 includes a relatively complex mix of different materials and a gas-impermeable portion to direct the released carbon monoxide towards the skin. The construction and the use of a mix of different materials results in a relatively complex and relatively expensive production process. In addition, the construction and the use of a mix of different materials makes it challenging, or even impossible, to miniaturize the device to smaller sizes to allow the device to be inserted into a human body and/or an animal body, e.g., orally and/or rectally.

Preferably, the entire wall of the housing, e.g., about an entire circumference of the housing/receptacle, is permeable to the carbon monoxide emitted by the main substance, which may allow the carbon monoxide emitted by the main substance to be released from a relatively large surface of the receptacle. This may increase the therapeutic effectiveness of the receptacle.

Preferably, the receptacle is configured such that the activation substance can be introduced into the chamber from outside of the housing, in a state in which the main substance is already arranged within the chamber, such that the section of the housing through which the activation substance was introduced, e.g., via an introducing device, e.g., a hollow needle, provides a substantially liquid-tight and gas-tight seal after the introduction of the activation substance has been completed, e.g., after the introducing device, e.g., the hollow needle, has been withdrawn from the housing. In other words, the section of the housing through which the activation substance was introduced may be configured to reseal after the introduction of the activation substance has been completed, e.g., when the introducing device, e.g., the hollow needle, has been withdrawn from the housing.

The receptacle may include one or more designated sections which are configured to allow and/or facilitate introduction of the activation substance into the chamber and/or to provide a substantially liquid-tight and gas-tight seal after the introduction of the activation substance has been completed, e.g., when the introducing device, e.g., the hollow needle, has been withdrawn from the housing. For instance, the housing may include one or more designated sections configured to be punctured by an introducing device, preferably a hollow needle, to introduce the activation substance into the chamber and to reseal after the introducing device has been removed from the chamber. The one or more designated sections may include an increased volume of material, e.g., a greater thickness of material, compared with adjacent sections of the housing, e.g., to facilitate puncturing the housing through the one or more designated sections and/or to facilitate resealing of the one or more designated sections, once the respective introducing device has been removed from the chamber.

The receptacle may have a substantially smooth outer contour and/or a rounded outer contour. In other words, the receptacle may be free of sharp edges on the outer contour, preferably the entire outer contour, of the receptacle. This may facilitate, and reduce the subject's discomfort when introducing the receptacle into the subject's body, e.g., by ingesting, e.g., swallowing, the receptacle and/or introducing the receptacle rectally.

The housing may be configured as a single integral component. In this case, for instance, the main substance and the activation substance may be introduced into the chamber, which is entirely enclosed, via a section of the wall of the housing, e.g., by puncturing the wall with a hollow needle and injecting the main substance and the activation substance into the chamber. Alternatively, the housing may comprise a plurality of components, e.g., a main body and a lid, which are assembled.

Preferably, the entire receptacle may be made from a single material, e.g., silicone. Being made of a single material does not mean that the entire receptacle is constructed integrally as a single piece. Instead, being made of a single material means that only a single material, e.g., silicone, was employed during the manufacturing of the receptacle. This may further reduce the manufacturing efforts for manufacturing the receptacle and/or may further simplify the construction of the receptacle. This may also facilitate adapting the size of the receptacle, e.g., by reducing the size of the receptacle, to the respective application.

The activation substance may be chosen from a variety of suitable substances. The activation substance may be a liquid and/or a gas, at least at the time of applying the activation substance to activate the carbon monoxide releasing main substance. For instance, the activation substance may be FeCl₃, Ce(SO₄)₂ or H₂O₂, preferably with FeCl₃.

The main substance may be configured to emit carbon monoxide after being activated by, e.g., reacting with, the activation substance. The carbon monoxide releasing main substance may be chosen from a variety of suitable substances. The main substance may be a liquid and/or a gas and/or a solid, at least at the time of applying the activation substance to activate the carbon monoxide releasing main substance. The carbon monoxide releasing main substance is preferably a metal carbonyl compound, even more preferably a molybdenum carbonyl compound, most preferably a salt formation of MO(CO)₃(CNCH₂CO₂H)H₃, preferably Na₃Mo(CO)₃(CNCH₂CO₂)₃.

Although the present disclosure is focused on emitting carbon monoxide from the receptacle, the receptacle may be configured to emit any gas, in particular any type of gas which may be used for therapeutic purposes. The main substance and the activation substance may then be selected accordingly to provide the respective desired gas and the material of the wall of the housing may be selected accordingly to be at least partially permeable to the respective gas.

As mentioned above, the receptacle preferably includes only a single chamber. The activation substance may be introduced into the chamber from outside of the housing, in a state in which the main substance is already arranged within the chamber, into the single chamber such that the main substance and the activation substance may react upon insertion of the activation substance into the single chamber from outside of the housing.

The wall of the housing may be non-permeable to one or more of the following: solids, liquids, toxic substances, preferably at least to heavy metal atoms containing substances, and byproducts, preferably at least toxic by-products, from the reaction between the main substance and the activation substance.

Preferably, the receptacle is configured to release carbon monoxide in vivo and/or ex vivo. Preferably, the receptacle is configured to be inserted, preferably manually, into one or more body cavities of an animal and/or a human, e.g., a child and/or an adult, e.g., by oral administration and/or rectal administration, and/or into an ear of an animal and/or a human for treatment of, e.g., (but not limited to) otitis media or other inflammatory diseases where a targeted release of CO may be of therapeutic use, and/or into a vagina of an animal and/or a human. Preferably, the receptacle is configured to be ingested, e.g., swallowed, by a human and/or an animal.

Preferably, at least one section of the housing is configured to be pierced by an introducing device configured to introduce the activation substance into the chamber to activate the main substance. Preferably, the at least one section of the housing is configured to reseal, preferably in a substantially liquid-tight and/or gas-tight manner, after the introducing device has been withdrawn from the housing. The introducing device may include a hollow needle, e.g., a syringe with a hollow needle.

Preferably, the housing includes at least one main body and at least one lid. Preferably, the main body and the lid are configured to be connectable to each other, preferably in a substantially liquid-tight and/or gas-tight manner. The main body and the lid may be connectable to each other by a friction-type connection, a form lock connection, and/or a material-bond connection, e.g., by at least one adhesive, such as at least one silicone adhesive.

Preferably, the lid and/or the main body include(s) a protrusion which is configured to extend into the chamber, when the lid and the main body are connected to each other.

Preferably, the protrusion is configured as a septum which is pierceable by an introducing device configured to introduce the activation substance into the chamber to activate the main substance, wherein the protrusion is configured to reseal, preferably in a substantially liquid-tight and/or gas-tight manner, after the introducing device has been withdrawn from the protrusion. Configuring the septum of the receptacle configured to introduce the activation substance into the chamber as a protrusion which extends into the chamber, as opposed to extending externally away from the chamber, may prevent the protrusion from causing discomfort to the subject when the receptacle is inserted into the subject's body, e.g., rectally and/or orally, and/or generally interfering with applying or administering the receptacle. For instance, a protrusion which extends externally away from the chamber may also be disadvantageous, when applying the receptacle ex-vivo, e.g., for treating biological cells, preferably living cells, in a container. For instance, a protrusion which extends externally away from the chamber may increase the outer dimensions of the receptacle, which may make it more difficult to arrange the receptacle in relatively compact spaces, e.g., in a container which houses living cells which are to be treated by the carbon monoxide which is emitted by the receptacle. Moreover, configuring the septum of the receptacle configured to introduce the activation substance into the chamber as a protrusion which extends into the chamber may prevent, or at least reduce the risk of, damage to the septum which may cause inadvertent release of the carbon monoxide releasing main substance and/or the activation substance, once the activation substance has been inserted into the chamber of the receptacle, and/or which may cause one or more substances, e.g., bodily fluids, to enter the receptacle, e.g., through a damaged section of the septum.

The protrusion may have any shape. Preferably, the protrusion has a substantially circular cross-sectional shape in a cross-section which extends transversely to a longitudinal axis which extends into the chambers when the lid and the main body are connected to each other.

Preferably, the protrusion has rounded or chamfered edges, e.g., at an intersection between one or more side walls of the protrusion and one or more end faces, at which the protrusion terminates in the chamber along a longitudinal axis of the housing. This may facilitate assembly of the receptacle. In particular, this may facilitate assembling the lid and the main body, e.g., by preventing relatively sharp edges of the protrusion from catching on a surface of the lid or the main body during assembly of the lid and the main body, or at least reducing the risk thereof, which may make assembly more difficult or may even prevent assembly, and/or by preventing a portion of the receptacle from being damaged during assembly of the lid and the main body, or at least reducing the risk thereof.

Preferably, the protrusion has a length, which extends into the chamber and/or along a longitudinal axis of the lid and/or the main body, when the main body and the lid are connected to each other, of at least 1 mm, more preferably at least 1.2 mm, more preferably at least 1.4 mm, more preferably at least 1.6 mm, more preferably at least 1.8 mm, more preferably at least 2 mm, more preferably at least 2.2 mm, more preferably at least 2.4 mm, more preferably at least 2.6 mm, more preferably at least 2.8 mm, more preferably at least 3 mm, more preferably at least 3.2 mm, more preferably at least 3.4 mm, more preferably at least 3.6 mm, more preferably at least 3.8 mm, more preferably at least 4 mm, more preferably at least 4.2 mm, more preferably at least 4.4 mm, more preferably at least 4.6 mm, more preferably at least 4.8 mm, more preferably at least 5 mm, more preferably at least 5.2 mm, more preferably at least 5.4 mm, more preferably at least 5.6 mm, more preferably at least 5.8 mm, most preferably at least 6 mm. The above-identified values for the length of the protrusion may provide a balance between a sufficient length to allow the activation substance to be introduced into the chamber via the protrusion, configured as the septum, such that the point of introduction in the protrusion may reseal after the introduction of the activation substance has been completed, while limiting the amount of space the protrusion occupies in the chamber.

Preferably, the main body and/or the lid has/have a slot, which preferably extends annularly or semi-annularly about a longitudinal axis of the main body and/or the lid. Preferably, a section of the other of the main body and the lid is configured to be inserted at least partially into the slot for connecting the main body and the lid to each other. This may provide a more reliable and sturdy connection between the lid and main body. Moreover, the slot may provide a guiding means when assembling the lid and the main body, which may facilitate the assembly of the lid and the main body. Moreover, configuring a section of the other of the main body and the lid to be inserted at least partially into the slot for connecting the main body and the lid to each other may at least partially provide a seal between the main body and the lid.

Preferably, when the lid and the main body are connected to each other, the protrusion at least partially delimits the slot, preferably along an outer circumference of the protrusion. Preferably, the slot extends coaxially at least partially about the protrusion.

Preferably, the main body and/or the lid is/are made of a silicone, preferably a bio-compatible silicone, preferably a medical grade silicone.

Preferably, the entire lid is integrally formed. Alternatively, or additionally, the entire main body may be integrally formed. This may facilitate the manufacturing process for manufacturing the lid and/or the main body. Alternatively, or additionally, this may facilitate the assembly of the lid and main body. The lid and/or the main body may each be molded and/or cast from a single material, e.g., silicone.

Preferably, the lid and/or the main body has a Shore A hardness from 20 Shore A to 80 Shore A, preferably from 30 Shore A to 50 Shore A.

Preferably, the main body and/or the lid include(s) at least one sealing and/or friction enhancing element, which is preferably arranged along at least one connection interface between the main body and the lid to increase a sealing effect and/or friction between the main body and the lid, when the main body and the lid are connected to each other. This may provide and/or increase a force, e.g., an adhering force and/or a frictional force, which acts between the main body and the lid to increase a sealing effect and/or friction between the main body and the lid, when the main body and the lid are connected to each other. Alternatively, or additionally, the sealing and/or friction enhancing element may increase an area of engagement and/or an area of adhesion between the main body and the lid. This may reduce leakage and/or a risk of leakage from the receptacle via an interface between the main body and the lid and/or may prevent, or at least reduce the risk of, unwanted disconnection or at least partial disconnection of the lid and the body.

Preferably, the sealing and/or friction enhancing element includes at least one projection which extends from an interface surface of the lid and/or the body. The interface surface may extend along a connection interface between the lid and the body. The projection may extend towards and may engage the other of the lid and the body, when the main body and the lid are connected to each other. The projection may increase a force which acts between the lid and the body to increase a sealing effect between the lid and the body and/or to increase a connection force between the lid and the body, e.g., to prevent, or at least reduce the risk of, unwanted disconnection or at least partial disconnection of the lid and the body. The projection may provide, or increase, a press-fit between the lid and the body.

Preferably, the lid or the body includes the projection. An interface surface of the other of the lid and the body, which extends along a connection interface between the lid and the body, may be substantially smooth and/or continuous and/or free of projections and/or free of recesses. This may increase a force which acts between the lid and the body. Alternatively, the other of the lid and the body may include at least one recess, e.g., at least one recess configured to at least partially receive the projection, and/or at least one projection.

Preferably, the projection extends at least partially along a circumference, preferably along the entire circumference, preferably multiple times, of the interface surface of the lid and/or the body, preferably in a spiraling manner and/or a thread-like manner and/or a meandering manner and/or a zig-zag manner. This may provide a relatively large surface along which the projection may provide an increased sealing effect and/or increased friction. The circumference may be an inner circumference and/or an outer circumference.

Preferably, the projection may be made of the same material as the rest of the lid and/or the protrusion and/or the main body, respectively. Preferably, the projection and the protrusion are integrally and/or monolithically formed, by manufacturing the lid and/or the main body, with the respective projection(s), in a single molding process.

Preferably, the lid includes the protrusion. The distal end of the protrusion, which extends furthest into the main body when the lid and the main body are connected to each other, may have one or more rounded and/or chamfered edges. Alternatively, or additionally, the protrusion may be tapered, at least along a section of the protrusion, towards a distal end of the protrusion, which extends furthest into the main body, when the lid and the main body are connected to each other. This may facilitate manufacturing the lid, e.g., by enabling an easier removal of the molded lid from a mold compared to a cylindrical shape of the protrusion and/or reducing a risk of damage during the removal, if the lid is manufactured by a molding process. In addition, this may avoid, or at least reduce, spillage of a substance, e.g., the activation substance, after removal of an introducing device, e.g., a hollow needle, for introducing the substance into the receptacle, e.g., by leaving a space, or an additional space, at the edge(s) of the distal end of the protrusion compared to configuring the protrusion with one or more sharp edges at the distal end.

Preferably, the entire receptacle has a maximum dimension, preferably a length which extends along a longitudinal axis of the receptacle, of no more than 25 mm, preferably no more than 24 mm, more preferably no more than 23 mm, more preferably no more than 22 mm, more preferably no more than 21 mm, more preferably no more than 20 mm, more preferably no more than 19 mm, more preferably no more than 18 mm, more preferably no more than 17 mm, more preferably no more than 16 mm, more preferably no more than 15 mm, more preferably no more than 14 mm, more preferably no more than 13 mm, more preferably no more than 12 mm. Configuring the receptacle to be relatively compact, per the above-identified maximum dimensions, may facilitate introducing the receptacle into a human body and/or an animal body, in particular via one or more natural orifices in the human body and/or the animal body, e.g., orally and/or rectally. Moreover, providing a relatively compact receptacle may allow the receptacle to be arranged in relatively compact spaces, at least more easily and/or flexibly, e.g., in a container which houses biological cells, preferably living cells, which are to be treated by the carbon monoxide which is emitted by the receptacle.

Preferably, the entire receptacle has a maximum dimension in a direction which extends substantially transverse to a longitudinal axis of the main body and/or a longitudinal axis of the lid of no more than 12 mm, more preferably no more than 11 mm, more preferably no more than 10 mm, more preferably no more than 9 mm, more preferably no more than 8 mm, more preferably no more than 7 mm, more preferably no more than 6 mm, more preferably no more than 5 mm, more preferably no more than 4 mm. As specified above, configuring the receptacle to be relatively compact, per the above-identified maximum dimensions of the receptacle in a direction which extends substantially transverse to a longitudinal axis of the main body and/or a longitudinal axis of the lid, may facilitate introducing the receptacle into a human body and/or an animal body, in particular via one or more natural orifices in the human body and/or the animal body, e.g., orally and/or rectally, and/or may facilitate arranging the receptacle in relatively compact spaces, at least more easily and/or flexibly, e.g., in a container which houses living cells which are to be treated by the carbon monoxide.

Preferably, the main body has at least one main body wall which at least partially defines the chamber. Preferably, the main body wall is at least partially permeable to the carbon monoxide which is emitted by the main substance after the main substance has been activated by the activation substance, and wherein the main body wall has a thickness ranging from 0.4 mm to 2.5 mm, preferably from 0.5 mm to 2.4 mm, more preferably from 0.5 mm to 2.3 mm, more preferably from 0.5 mm to 2.2 mm, more preferably from 0.5 mm to 2.1 mm, more preferably from 0.5 mm to 2 mm, more preferably from 0.5 mm to 1.9 mm, more preferably from 0.5 mm to 1.8 mm, more preferably from 0.5 mm to 1.7 mm, more preferably from 0.5 mm to 1.6 mm, more preferably from 0.5 mm to 1.5 mm, more preferably from 0.5 mm to 1.4 mm, more preferably from 0.5 mm to 1.3 mm, more preferably from 0.5 mm to 1.2 mm, more preferably from 0.5 mm to 1.1 mm, more preferably from 0.5 mm to 1 mm, more preferably from 0.5 mm to 0.9 mm. In any case, the values for the thickness of the main body wall recited above provide a balance between the build size of the receptacle (or the size of the chamber within the main body wall which may be reduced for greater main body wall thicknesses) and the effectiveness of the therapeutic effects provided by the receptacle.

The thickness of the main body wall may have an effect on the release characteristics of the carbon monoxide from the chamber via the main body wall. In particular, a thinner main body wall may allow the carbon monoxide to be released at a higher rate than a thicker main body wall, at least when using the same material for the main body wall. In this respect, the desired release rate of the carbon monoxide may depend on the respective application of the receptacle, e.g., the respective desired therapeutic effects, the total quantity of carbon monoxide to be released, and/or the type of therapeutic treatment to be performed. Thus, the values for the thickness of the main body wall recited above may be selected according to the desired application.

While it is described above that the septum/protrusion may be provided by a protrusion of the lid, such septum or protrusion may, alternatively, be provided at the bottom of the main body (e.g., where the main body is integrally formed but also where a lid is used) and/or at a side (wall) of the main body. In other words, the wall of the main body at the bottom and/or side may have a thickness that corresponds to the preferred protrusion lengths mentioned above.

Preferably, the main body has a bottom end which is arranged opposite from the lid with respect to a direction extending from the lid towards the bottom end, when the lid and the main body are connected to each other. Preferably, the bottom end has a convex outer shape. Configuring the bottom end to have a convex outer shape may facilitate, and/or reduce the subject's discomfort when, introducing the receptacle into the subject's body, e.g., by ingesting, e.g., swallowing, the receptacle and/or introducing the receptacle rectally.

Preferably, the lid has at least one flange and the main body has a main body wall which at least partially defines the chamber. Preferably, the flange is configured to extend along at least a section of the main body wall, when the lid and the main body are connected to each other. Such a configuration may provide a more reliable and sturdy connection between the lid and main body. Moreover, the flange may provide a guiding means for the main body wall when assembling the lid and the main body, which may facilitate the assembly of the lid and the main body. The flange may be configured as a collar which extends radially and/or axially, with respect to a longitudinal axis of the receptacle.

Preferably, the slot and the protrusion are provided on the lid, preferably integrally. Preferably, the slot is at least partially delimited by at least a portion of the flange and at least a portion of the protrusion, preferably such that a section of the main body, preferably a section of an annular wall of the main body, can be inserted at least partially between the protrusion and the flange for connecting the main body and the lid with each other. This may provide a more reliable and sturdy connection between the lid and main body. Moreover, the flange and the protrusion may provide a guiding means for at least a section of the wall of the main body, when assembling the lid and the main body, which may facilitate the assembly of the lid and the main body.

Preferably, the main body and the lid are connectable to each other by means of a material bond, preferably by means of an adhesive, preferably an adhesive made of a silicone, preferably a bio-compatible silicone, preferably a medical grade silicone.

Preferably, the entire receptacle has a size which corresponds to, or is at least not larger than, a standard drug capsule size, preferably a standard drug capsule size from 5 to 000.

Preferably, the entire receptacle has a size which corresponds to, or is at least not larger than, a standard drug capsule size of 2, 1, or 0.

Preferably, the main body and/or the lid is/are manufactured by means of a molding process, preferably by a compression molding process and/or an injection molding process.

A second aspect of the present disclosure relates to a device for selectively releasing carbon monoxide, including the receptacle according to any of the configurations disclosed herein. The device may further include at least one main substance configured to selectively emit carbon monoxide. The main substance may be stored within the chamber of the receptacle and may be configured to emit carbon monoxide such that the emitted carbon monoxide is released through at least a section of the wall of the receptacle after being activated by at least one activation substance.

The configurations and advantages, as detailed above with respect to the receptacle, apply to the device accordingly.

Preferably, the entire device, according to the second aspect of the present disclosure, may be configured as a capsule.

A third aspect of the present disclosure relates to a treatment system for treating biological cells, preferably living cells, preferably immune cells, such as CAR T cells and/or stem cells, and/or gametes, with carbon monoxide, which may include at least one container configured to receive the biological cells and at least one source of carbon monoxide, preferably at least one device for selectively releasing carbon monoxide according to the second aspect of the present disclosure. The container and the source of carbon monoxide may be arranged relative to each other such that the biological cells may be contacted by the carbon monoxide provided by the source of carbon monoxide to treat the biological cells.

The configurations and advantages, as detailed above with respect to the receptacle, apply to the treatment system accordingly.

The treatment system may be configured to provide one or more effects to the biological cells which at least partially preserve the biological cells.

Preferably, the biological cells are immune cells, such as CAR T cells and/or stem cells, and/or gametes. The gametes preferably comprise spermatozoa or egg cells, preferably wherein the spermatozoa are comprised within seminal fluid, ejaculate and/or a buffer system.

The biological cells may be human gametes and/or human immune cells. Alternatively, or additionally, the gametes and/or immune cells may be from animals, preferably from cattle, horses, pigs, sheep, goats, camels, alpacas, dogs, and/or cats.

The container may be configured to receive, preferably entirely receive, the biological cells and the source of carbon monoxide.

The container may include at least one compartment configured to receive, and preferably house and/or secure, preferably in a captive manner, the carbon monoxide releasing device. This may allow the treatment system to be preassembled, in particular by arranging the carbon monoxide releasing device in the compartment prior to use, preferably prior to being distributed to the locations of application, e.g., one or more laboratories. This may facilitate handling and/or use of the treatment system and/or may prevent the carbon monoxide releasing device from being lost and/or separated from the container, e.g., during transport and/or handling.

The biological cells and the device for releasing carbon monoxide may be placed in a single compartment within the container.

Alternatively, the biological cells and the device for releasing carbon monoxide may be placed in separate compartments of the container, the compartments preferably being separated from each other by at least one wall and/or barrier which is permeable with respect to the carbon monoxide. The wall and/or barrier may have one or more openings configured to allow the carbon monoxide to pass therethrough to the biological cells. Preferably, the compartment for receiving the carbon monoxide releasing device is arranged at and/or along a bottom of the container, in particular such that the biological cells are arranged at least partially above said compartment, in particular on an upper wall of said compartment. This may increase a degree of exposure and/or an efficiency of exposure of the biological cells to the carbon monoxide released by the device. Alternatively, the compartment may be arranged at and/or along a top and/or a side of the container.

The container may include a lid, which is preferably configured to provide a seal with the container to seal the contents of the container from an environment, preferably in a gas-tight manner.

A fourth aspect of the present disclosure relates to a set, preferably for treating biological cells, preferably living cells, ex-vivo or in-vivo with carbon monoxide. The set includes at least one device for selectively releasing carbon monoxide according to the second aspect of the present disclosure. The set may further include at least one activation substance, preferably wherein the activation substance is contained within, or at least configured to be provided within, an introducing device with a hollow needle configured to pierce through the device for selectively releasing carbon monoxide. More preferably, the hollow needle is configured to pierce through a lid of the device for selectively releasing carbon monoxide. The introducing device may be part of the set.

The configurations and advantages, as detailed above with respect to the receptacle, apply to the set accordingly.

A fifth aspect of the present disclosure relates to a method of treating a mammal, such as a human, in-vivo with carbon monoxide. The method may comprise providing at least one device for selectively releasing carbon monoxide according to the second aspect of the present disclosure. The method may further comprise activating the main substance with at least one activation substance, such as by introducing the activation substance into a chamber of the device through a lid or a main body of the device. The method may further comprise introducing the at least one device into the mammal, preferably into one or more body cavities of the mammal, such as by oral ingestion and/or rectally, and/or into an ear of an animal and/or a human for treatment of, e.g., (but not limited to) otitis media or other inflammatory diseases where a targeted release of CO may be of therapeutic use, and/or into a vagina of an animal and/or a human.

The configurations and advantages, as detailed above with respect to the receptacle, apply to the method accordingly.

A sixth aspect of the present disclosure relates to carbon monoxide for use in treating biological cells, preferably living cells, in and/or outside of a mammal's body, preferably for treating a gastrointestinal disease. Preferably, the carbon monoxide is released from a device for selectively releasing carbon monoxide, preferably from a device according to any of the embodiments described herein (see the second aspect above), within the mammal's body and/or outside of the mammal's body.

The configurations and advantages, as detailed above with respect to the receptacle, apply to the sixth aspect accordingly.

A seventh aspect of the present disclosure relates to a method of treating biological cells, preferably living cells, more preferably ex vivo, with carbon monoxide. The method may comprise providing a treatment system according to the above-described third aspect. The method may comprise arranging the biological cells in the container. The method may comprise triggering a release of the carbon monoxide from the source of carbon monoxide. The carbon monoxide provided by the source of carbon monoxide may contact the biological cells.

The configurations and advantages, as detailed above, apply to the seventh aspect accordingly.

Preferably, the treatment system is provided by arranging at least one device configured to selectively release carbon monoxide in the container, preferably at least one device according to any of the embodiments described herein with respect to the second aspect as described above.

Preferably, triggering the release of carbon monoxide comprises activating a main substance of the source of carbon monoxide with at least one activation substance.

Preferably, the activation substance is introduced into a chamber in which the main substance is arranged. Preferably, the activation substance is introduced via an introducing device. The introducing device may comprise a hollow needle configured to pierce through a wall of the chamber.

Preferably, the treatment system comprises at least one first chamber and at least one second chamber. The biological cells may be arranged in a first chamber. At least a main substance may be arranged in the second chamber. The second chamber may be separated from the first chamber by at least one wall. At least a section of the wall may be permeable to carbon monoxide emitted by the main substance such that carbon monoxide emitted by the main substance is released from the second chamber into the first chamber.

Preferably, the carbon monoxide is released from the device in at least a section of the mammal's gastrointestinal tract, e.g., for treating a gastrointestinal disease, e.g., an inflammatory bowel disease such as ulcerative colitis and/or Crohn's disease.

Preferably, the device is introduced enterally, preferably by ingestion and/or rectally, into the mammal's body.

The following list of aspects provides preferred embodiments of the present disclosure:
1. A receptacle, which is preferably configured to be inserted into a human body and/or an animal body, including:
   at least one housing;
   at least one chamber defined within the housing;
   wherein the chamber is configured to receive and store at least one main substance configured to selectively emit at least one gas, preferably at least carbon monoxide, wherein the main substance is configured to emit the gas after being activated by at least one activation substance;
   wherein the chamber is enclosed by at least one wall of the housing, wherein at least a section of the wall is at least partially permeable to the gas emitted by the main substance such that the receptacle can at least partially release the gas emitted by the main substance to an environment; and
   wherein the receptacle is preferably configured such that the activation substance can be introduced into the chamber from outside of the housing, in a state in which the main substance is already arranged within the chamber.
2. The receptacle according to aspect 1, wherein the receptacle includes only a single chamber.
3. The receptacle according to aspect 1 or 2, wherein the wall of the housing is non-permeable to one or more the following: solids, liquids, toxic substances, preferably at least to heavy metal atoms containing substances, and byproducts, preferably at least toxic byproducts, from the reaction between the main substance and the activation substance.
4. The receptacle according to any of the preceding aspects, wherein the receptacle is configured to release the gas, preferably carbon monoxide, in vivo and/or ex vivo.
5. The receptacle according to any of the preceding aspects, wherein the receptacle is configured to be inserted into a human body and/or an animal body.
6. The receptacle according to any of the preceding aspects, wherein the receptacle is configured to be administered orally and/or rectally into a human body and/or an animal body.
7. The receptacle according to any of the preceding aspects, wherein the receptacle is configured to be ingested, preferably swallowed, by a human and/or an animal.
8. The receptacle according to any of the preceding aspects, wherein at least one section of the housing is configured to be pierced by an introducing device configured to introduce the activation substance into the chamber to activate the main substance, wherein the at least one section of the housing is configured to reseal, preferably in a substantially liquid-tight and/or gas-tight manner, after the introducing device has been withdrawn from the housing.
9. The receptacle according to any of the preceding aspects, wherein the housing includes at least one main body and at least one lid, wherein the main body and the lid are configured to be connectable to each other, preferably in a substantially liquid-tight and/or gas-tight manner.
10. The receptacle according to aspect 9, wherein the lid and/or the main body include(s) a protrusion which is configured to extend into the chamber, when the lid and the main body are connected to each other.
11. The receptacle according to aspect 10, wherein the protrusion is configured as a septum which is pierceable by an introducing device configured to introduce the activation substance into the chamber to activate the main substance, wherein the protrusion is configured to reseal, preferably in a substantially liquid-tight and/or gas-tight manner, after the introducing device has been withdrawn from the protrusion.
12. The receptacle according to aspect 10 or 11, wherein the protrusion has a length, which extends into the chamber and/or along a longitudinal axis of the lid and/or the main body, when the main body and the lid are connected to each other, of at least 1 mm, more preferably at least 1.2 mm, more preferably at least 1.4 mm, more preferably at least 1.6 mm, more preferably at least 1.8 mm, more preferably at least 2 mm, more preferably at least 2.2 mm, more preferably at least 2.4 mm, more preferably at least 2.6 mm, more preferably at least 2.8 mm, more preferably at least 3 mm, more preferably at least 3.2 mm, more preferably at least 3.4 mm, more preferably at least 3.6 mm, more preferably at least 3.8 mm, more preferably at least 4 mm, more preferably at least 4.2 mm, more preferably at least 4.4 mm, more preferably at least 4.6 mm, more preferably at least 4.8 mm, more preferably at least 5 mm, more preferably at least 5.2 mm, more preferably at least 5.4 mm, more preferably at least 5.6 mm, more preferably at least 5.8 mm, most preferably at least 6 mm.
13. The receptacle according to any of aspects 9 to 12, wherein the main body and/or the lid has/have a slot, which preferably extends annularly or semi-annularly about a longitudinal axis of the main body and/or the lid, wherein a section of the other of the main body and the lid is configured to be inserted at least partially into the slot for connecting the main body and the lid to each other.
14. The receptacle according to aspect 13, wherein, when the lid and the main body are connected to each other, the protrusion at least partially delimits the slot, preferably along an outer circumference of the protrusion, wherein preferably the slot extends coaxially at least partially about the protrusion.
15. The receptacle according to any of aspects 9 to 14, wherein the main body and/or the lid is/are made of a silicone, preferably a bio-compatible silicone, preferably a medical grade silicone.
16. The receptacle according to any of aspects 9 to 15, wherein the entire lid is integrally formed.
17. The receptacle according to any of aspects 9 to 16, wherein the entire main body is integrally formed.
18. The receptacle according to any of aspects 9 to 17, wherein the lid and/or the main body has a Shore A hardness from 20 Shore A to 80 Shore A, preferably from 30 Shore A to 50 Shore A.
19. The receptacle according to any of aspects 9 to 18, wherein the main body and/or the lid include(s) at least one sealing and/or friction enhancing element arranged along at least one connection interface between the main body and the lid to increase a sealing effect and/or friction between the main body and the lid, when the main body and the lid are connected to each other.
20. The receptacle according to aspect 19, wherein the sealing and/or friction enhancing element includes at least one projection (e.g., rib) which extends from an interface surface of the lid and/or the body, the interface surface extending along the connection interface, wherein the projection extends towards and engages the other of the lid and the body, when the main body and the lid are connected to each other.
21. The receptacle according to aspect 20, wherein the lid or the body includes the projection and an interface surface of the other of the lid and the body, which extends along a connection interface between the lid and the body, is substantially smooth and/or continuous and/or free of projections and/or free of recesses.
22. The receptacle according to aspect 20 or 21, wherein the projection extends at least partially along a circumference, preferably along the entire circumference, preferably multiple times, of the interface surface of the lid and/or the body, preferably in a spiraling manner and/or a thread-like manner and/or a meandering manner and/or a zig-zag manner.
23. The receptacle according to any of aspects 9 to 22, wherein the lid includes the protrusion, and wherein:
   a distal end of the protrusion, which extends furthest into the main body when the lid and the main body are connected to each other, has one or more rounded and/or cambered edges;
   and/or
   the protrusion is tapered, at least along a section of the protrusion, towards a distal end of the protrusion, which extends furthest into the main body when the lid and the main body are connected to each other.
24. The receptacle according to any of aspects 20 to 23, wherein the projection and the protrusion are integrally and/or monolithically formed.
25. The receptacle according to any of the preceding aspects, wherein the entire receptacle has a maximum dimension, preferably a length which extends along a longitudinal axis of the receptacle, of no more than 25 mm, preferably no more than 24 mm, more preferably no more than 23 mm, more preferably no more than 22 mm, more preferably no more than 21 mm, more preferably no more than 20 mm, more preferably no more than 19 mm, more preferably no more than 18 mm, more preferably no more than 17 mm, more preferably no more than 16 mm, more preferably no more than 15 mm, more preferably no more than 14 mm, more preferably no more than 13 mm, more preferably no more than 12 mm.
26. The receptacle according to any of the preceding aspects, wherein the entire receptacle has a maximum dimension in a direction which extends substantially transverse to a longitudinal axis of the main body and/or a longitudinal axis of the lid of no more than 12 mm, more preferably no more than 11 mm, more preferably no more than 10 mm, more preferably no more than 9 mm, more preferably no more than 8 mm, more preferably no more than 7 mm, more preferably no more than 6 mm, more preferably no more than 5 mm, more preferably no more than 4 mm.
27. The receptacle according to any of aspects 9 to 26, wherein the main body has at least one main body wall which at least partially defines the chamber, wherein the main body wall is at least partially permeable to the gas which is emitted by the main substance after the main substance has been activated by the activation substance, and wherein the main body wall has a thickness ranging from 0.4 mm to 2.5 mm, preferably from 0.5 mm to 2.4 mm, more preferably from 0.5 mm to 2.3 mm, more preferably from 0.5 mm to 2.2 mm, more preferably from 0.5 mm to 2.1 mm, more preferably from 0.5 mm to 2 mm, more preferably from 0.5 mm to 1.9 mm, more preferably from 0.5 mm to 1.8 mm, more preferably from 0.5 mm to 1.7 mm, more preferably from 0.5 mm to 1.6 mm, more preferably from 0.5 mm to 1.5 mm, more preferably from 0.5 mm to 1.4 mm, more preferably from 0.5 mm to 1.3 mm, more preferably from 0.5 mm to 1.2 mm, more preferably from 0.5 mm to 1.1 mm, more preferably from 0.5 mm to 1 mm, more preferably from 0.5 mm to 0.9 mm
28. The receptacle according to any of aspects 9 to 27, wherein the main body has a bottom end which is arranged opposite from the lid with respect to a direction extending from the lid towards the bottom end, when the lid and the main body are connected to each other, wherein the bottom end has a convex outer shape.
29. The receptacle according to any of aspects 9 to 28, wherein the lid has at least one flange and the main body has a main body wall which at least partially defines the chamber, wherein the flange is configured to extend along at least a section of the main body wall when the lid and the main body are connected to each other.
30. The receptacle according to any of aspects 9 to 29, wherein the slot and the protrusion are provided on the lid, preferably integrally, wherein the slot is at least partially delimited by at least a portion of the flange and at least a portion of the protrusion, preferably such that a section of the main body, preferably a section of an annular wall of the main body, can be inserted at least partially between the protrusion and the flange for connecting the main body and the lid with each other.
31. The receptacle according to any of aspects 9 to 30, wherein the main body and the lid are connectable to each other by means of a material bond, preferably by means of an adhesive, preferably an adhesive made of a silicone, preferably a bio-compatible silicone, preferably a medical grade silicone.
32. The receptacle according to any of the preceding aspects, wherein the entire receptacle has a size which corresponds to, or is at least not larger than, a standard drug capsule size, preferably a standard drug capsule size from 5 to 000.
33. The receptacle according to any of the preceding aspects, wherein the entire receptacle has a size which corresponds to, or is at least not larger than, a standard drug capsule size of 2, 1, or 0.
34. The receptacle according to any of the preceding aspects, wherein the main body and/or the lid is/are manufactured by means of a molding process, preferably by a compression molding process and/or an injection molding process.
35. A device for selectively releasing at least one gas, preferably at least carbon monoxide, including:
   the receptacle according to any of the preceding aspects; and
   at least one main substance configured to selectively emit the gas, wherein the main substance is stored within the chamber of the receptacle and is configured to emit the gas such that the emitted gas is released through at least a section of the wall of the receptacle after being activated by at least one activation substance.
36. A treatment system for treating biological cells, preferably living cells, preferably immune cells and/or gametes, with at least one gas, preferably at least carbon monoxide, including:
   at least one container configured to receive the biological cells;
   at least one source of the gas, preferably of carbon monoxide, preferably at least one device for selectively releasing at least one gas, preferably at least carbon monoxide, according to aspect 35;
   wherein the container and the source of the gas are arranged relative to each other such that the biological cells may be contacted by the gas provided by the source of the gas to treat the biological cells.
37. The treatment system according to aspect 36, wherein the container is configured to receive, preferably entirely, the biological cells and the source of the gas.
38. A set for treating biological cells, preferably living cells, ex-vivo or in-vivo with at least one gas, preferably at least carbon monoxide, the set including:
   at least one device for selectively releasing at least one gas, preferably at least carbon monoxide, according to aspect 35; and
   at least one activation substance, preferably wherein the activation substance is contained within an introducing device with a hollow needle configured to pierce through the device for selectively releasing the gas, more preferably wherein the hollow needle is configured to pierce through a lid of the device for selectively releasing the gas.
39. A method of treating a mammal, such as a human, in-vivo with at least one gas, preferably at least carbon monoxide, the method comprising:
   providing at least one device for selectively releasing at least one gas, preferably at least carbon monoxide, according to aspect 35;
   activating the main substance with at least one activation substance, such as by introducing the activation substance into a chamber of the device through a lid or a main body of the device;
   introducing the at least one device into the mammal, such as by oral ingestion and/or rectal ingestion and/or and/or into an ear of the mammal and/or into a vagina of the mammal.
40. Carbon monoxide for use in treating biological cells, preferably living cells, in and/or outside of a mammal's body, preferably for treating a gastrointestinal disease, preferably an inflammatory disease of the gastrointestinal tract, or a gastrointestinal transplant, wherein the carbon monoxide is released from a device according to aspect 35 within the mammal's body and/or outside of the mammal's body.
41. Carbon monoxide according to aspect 40, wherein the carbon monoxide is released from the device in at least a section of the mammal's gastrointestinal tract.
42. Carbon monoxide according to aspect 40 or 41, wherein the device is introduced enterally, preferably by ingestion and/or rectally and/or into an ear of the mammal and/or into a vagina of the mammal, into the mammal's body.
43. Carbon monoxide according to aspect 40, 41, or 42, wherein the disease is one or more of: diabetic gastroparesis, post-operative ileus, inflammatory bowel disease (such as ulcerative colitis and/or Crohn's disease), sepsis, gastritis, gastric ulcers, ischemia-reperfusion injuries of the gastrointestinal tract, non-alcoholic steatohepatitis.
44. A method of treating biological cells, preferably living cells, more preferably ex vivo, with carbon monoxide, the method comprising:
   providing a treatment system according to aspect 36;
   arranging the biological cells in the container;
   triggering a release of the carbon monoxide from the source of carbon monoxide, wherein the carbon monoxide provided by the source of carbon monoxide contacts the biological cells.
45. The method of aspect 44, wherein the treatment system is provided by arranging at least one device configured to selectively release carbon monoxide in the container, preferably at least one device according to aspect 35.
46. The method of aspect 44 or 45, wherein triggering the release of carbon monoxide comprises activating a main substance of the source of carbon monoxide with at least one activation substance.
47. The method of aspect 46, wherein the activation substance is introduced into a chamber in which the main substance is arranged, preferably wherein the activation substance is introduced via an introducing device, wherein the introducing device comprises a hollow needle configured to pierce through a wall of the chamber.
48. The method of any of aspects 44 to 47, wherein the treatment system comprises at least one first chamber and at least one second chamber, wherein the biological cells are arranged in a first chamber, and wherein at least a main substance is arranged in the second chamber, preferably wherein the second chamber is separated from the first chamber by at least one wall, preferably wherein at least a section of the wall is permeable to the carbon monoxide emitted by the main substance such that carbon monoxide emitted by the main substance is released from the second chamber into the first chamber.

Preferred embodiments of the present invention are further elucidated below with reference to the figures. The described embodiments do not limit the present invention.

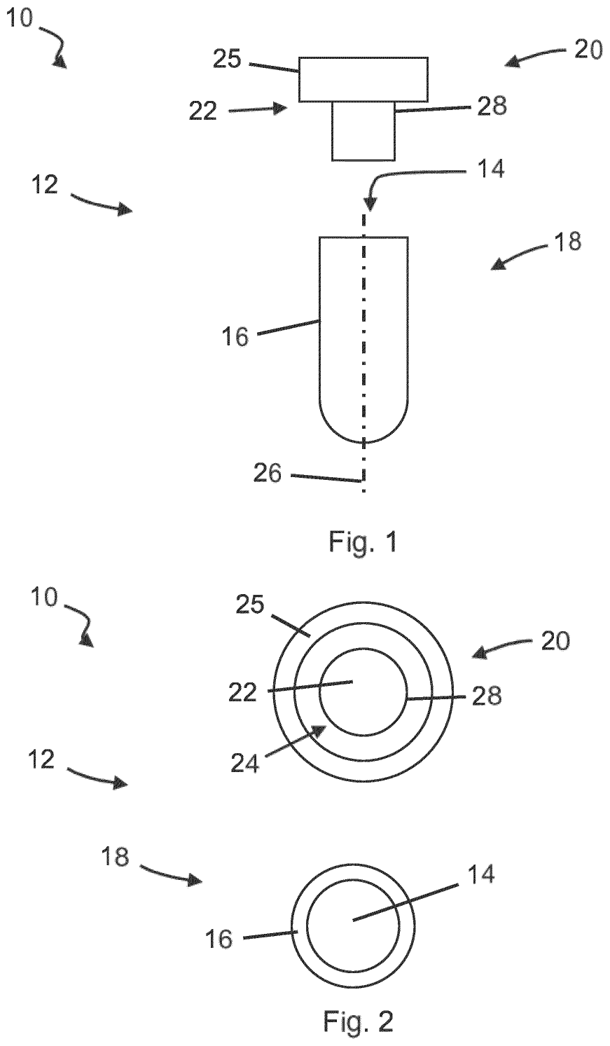

FIG. 1 shows, in a schematic illustration, an exploded view of a receptacle according to an embodiment of the present disclosure;

FIG. 2 shows, in a schematic illustration, a further exploded view of the receptacle shown in FIG. 1;

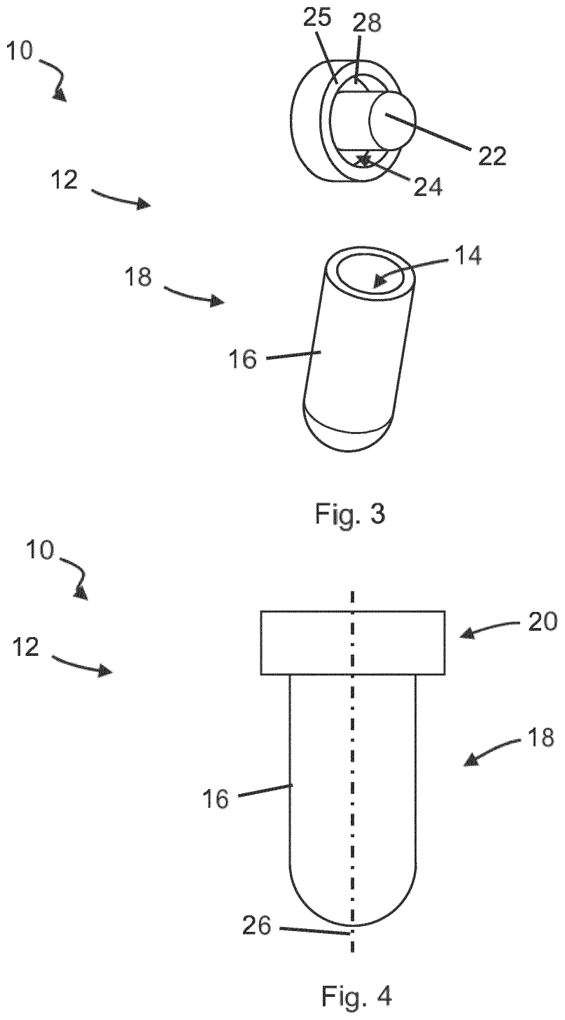

FIG. 3 shows, in a schematic illustration, a further exploded view of the receptacle shown in FIGS. 1 and 2;

FIG. 4 shows, in a schematic illustration, the receptacle of FIGS. 1 to 3 in an assembled state;

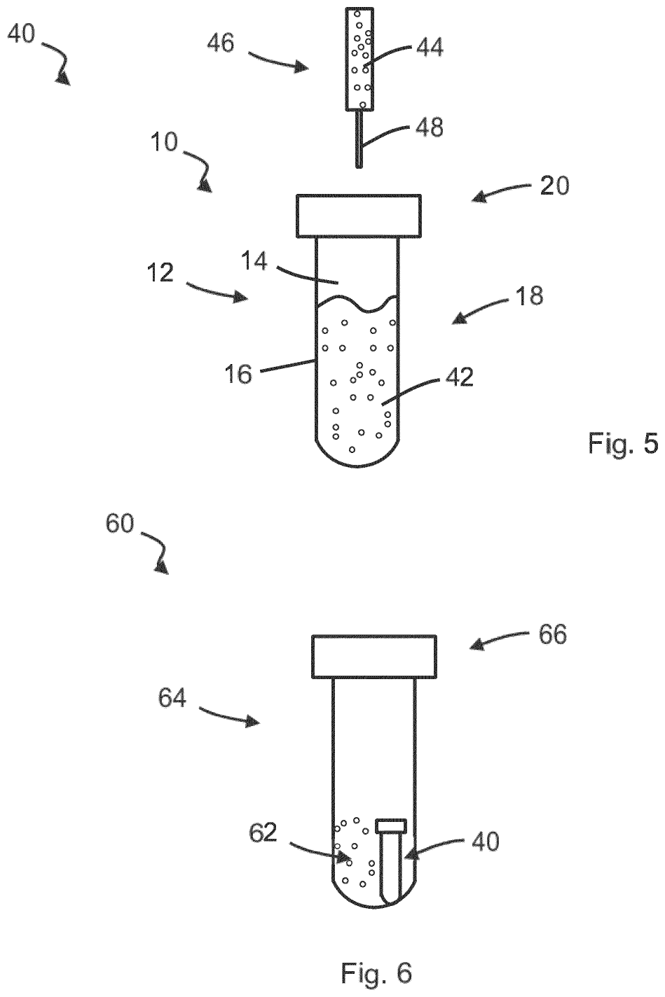

FIG. 5 shows, in a schematic illustration, a side view of a device for selectively releasing carbon monoxide which includes the receptacle shown in FIGS. 1 to 4;

FIG. 6 shows, in a schematic illustration, a side view of a treatment system according to an embodiment of the present disclosure;

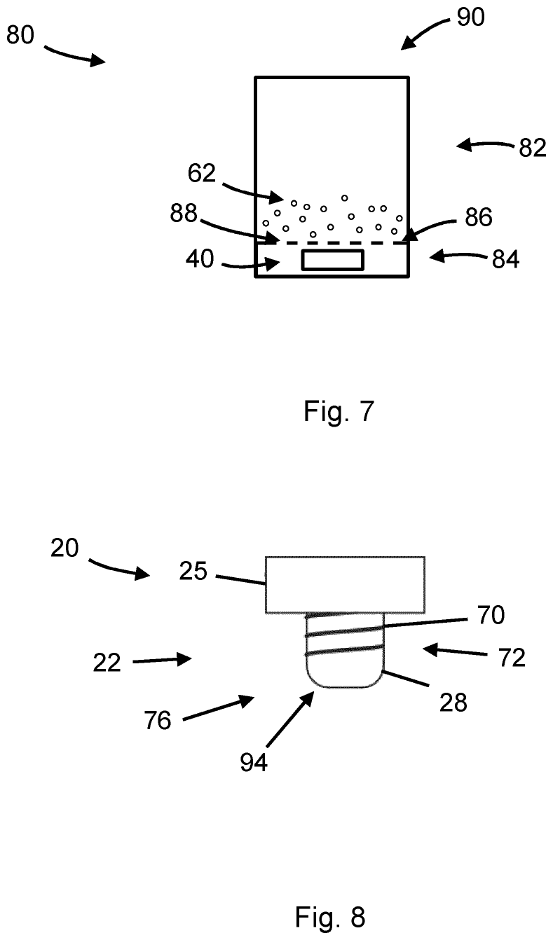

FIG. 7 shows, in a schematic illustration, a side view of a treatment system according to a further embodiment of the present disclosure;

FIG. 8 shows, in a schematic illustration, a modification of the lid of the receptacle shown in FIGS. 1 to 6;

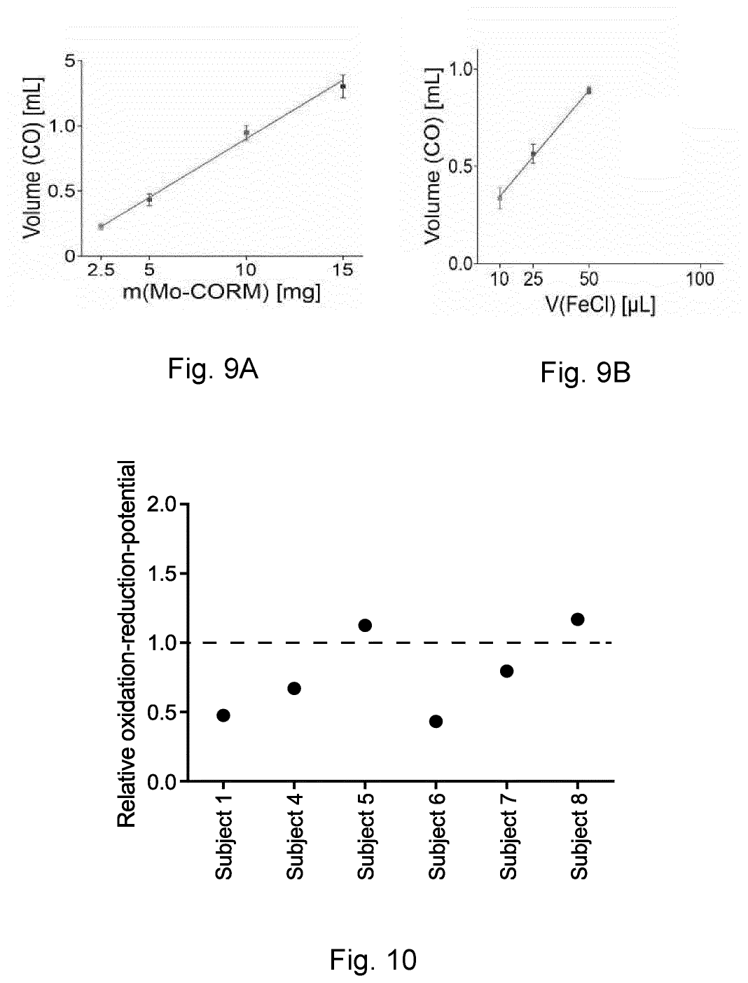

FIG. 9A shows a correlation between a released volume of carbon monoxide from the receptacle of FIGS. 1 to 8 and a mass of the main substance;

FIG. 9B shows a correlation between a released volume of carbon monoxide from the receptacle of FIGS. 1 to 8 and a volume of the activation substance;

FIG. 10 shows, in a schematic diagram, results of an exemplary treatment of spermatozoa with carbon monoxide gas;

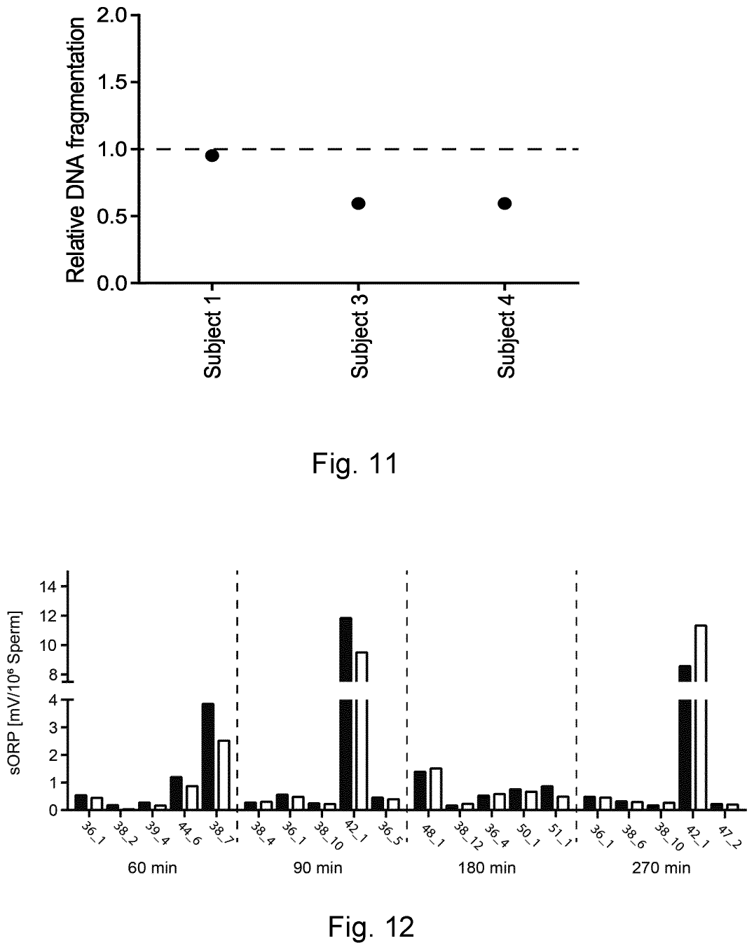

FIG. 11 shows, in a further schematic diagram, results of an exemplary treatment of spermatozoa with carbon monoxide gas;

FIG. 12 shows, in a further schematic diagram, results of a further exemplary treatment of spermatozoa with carbon monoxide gas.

FIGS. 1 to 3 show exploded views of a receptacle 10 according to an embodiment of the present disclosure. FIG. 4 shows the receptacle 10 of FIGS. 1 to 3 in an assembled state. The receptacle 10 may be configured to be inserted into a human body and/or an animal body. Preferably, the receptacle 10 is configured to be inserted, preferably manually, e.g., by being administered orally, e.g., by ingesting, such as by swallowing, the receptacle 10, and/or rectally and/or into an ear and/or a vagina of the animal and/or a human, into one or more body cavities of a human body and/or an animal body.

The receptacle 10 may include at least one housing 12 and at least one chamber 14 defined within the housing 12. The chamber 14 may be configured to receive and store at least one main substance configured to selectively emit carbon monoxide. In particular, the main substance may be configured to emit carbon monoxide after being activated by at least one activation substance.

The chamber 14 may be enclosed by at least one wall 16 of the housing 12. At least a section of the wall 16 may be at least partially permeable to the carbon monoxide emitted by the main substance such that the receptacle 10 can at least partially release the carbon monoxide emitted by the main substance to an environment, e.g., a treatment site, e.g., within the subject's body. The receptacle 10 may be configured such that the activation substance can be introduced into the chamber 14 from outside of the housing 12, e.g., by means of an introducing device, such as a syringe which has a hollow needle configured to puncture a portion of the housing 12, in a state in which the main substance is already arranged within the chamber 14.

The wall 16 of the housing 12 may be non-permeable to one or more of the following: solids, liquids, toxic substances, preferably at least to heavy metal atoms containing substances, and by-products, preferably at least toxic by-products, from the reaction between the main substance and the activation substance.

As shown in FIGS. 1 to 3, the receptacle 10 may include only a single chamber 14. This may allow the receptacle 10 to be relatively compact. However, alternatively, the receptacle 10 may include a plurality of chambers 14.

The housing 12 may include at least one main body 18 and at least one lid 20. The main body 18 and the lid 20 may be configured to be connected to each other, preferably in a substantially liquid-tight and/or gas-tight manner, such that the chamber 14 is substantially enclosed entirely against the environment, preferably in a substantially liquid-tight and/or gas-tight manner. The main body 18 and the lid 20 may be connectable to each other, e.g., by a friction-type connection, a form lock connection, and/or a material-bond connection, e.g., by at least one adhesive, such as at least one silicone adhesive. The main body 18 and/or the lid 20 may be made of silicone. Preferably, the entire receptacle 10 is made of a single material, e.g., silicone.

As shown in FIGS. 1 to 3, the lid 20 may include a protrusion 22 which is configured to extend into the chamber 14, when the lid 20 and the main body 18 are connected to each other.

The protrusion 22 may be configured as a septum which is pierceable by an introducing device configured to introduce the activation substance into the chamber 14 to activate the main substance. A suitable introducing device may be a syringe which has a hollow needle configured to pierce through the protrusion 22 and extend into the chamber 14 to deposit the activation substance in the chamber 14. Preferably, the protrusion 22 or septum is configured to reseal, preferably in a substantially liquid-tight and/or gas-tight manner, after the introducing device has been withdrawn from the protrusion 22.

Alternatively, or additionally, the main body 18 may include a protrusion and/or septum, which may be similar or identical to the protrusion 22 shown in FIGS. 1 to 3. The protrusion and/or septum provided on the main body may also be configured to extend into the chamber 14, or to at least be arranged in the chamber 14, when the lid 20 and the main body 18 are connected to each other. For example, the wall at the bottom of the main body 18 (e.g., at the end of the main body 18 opposite the lid 20) and/or at a side of the main body 18 may provide a septum. For this purpose, the wall at the bottom and/or the side may have a larger thickness than a peripheral (e.g., cylindrical) portion of the wall 16. The wall at the bottom and/or the side may have a thickness of at least 1.2 mm, preferably at least 2 mm, more preferably at least 3 mm, more preferably at least 4 mm, and even more preferably at least 5 mm.

As shown in FIGS. 1 to 3, the lid 20 may have a slot 24 (see FIGS. 2 and 3) which may extend annularly or semi-annularly about a longitudinal axis 26 of the main body 18 and/or the lid 20. The slot 24 may extend coaxially at least partially about the protrusion 22, as shown in FIGS. 1 to 3.

The lid 20 may include at least one flange 25 which may be configured to extend along at least a section of the wall 16, when the lid 20 and the main body 18 are connected to each other (see FIG. 4).

The slot 24 and the protrusion 22 may be provided on the lid 22, as shown in FIGS. 1 to 3, preferably integrally. The slot 24 may be at least partially delimited by at least a portion of the flange 25 and at least a portion of the protrusion 22, preferably along an outer circumference 28 of the protrusion 22, as shown in FIGS. 1 to 3. A section of the wall 16 can be inserted at least partially between the protrusion 22 and the flange 25, i.e., in the slot 24, to aid in connecting the main body 18 and the lid 20 with each other.

Alternatively, or additionally, the main body 18 may include a slot, which may be similar or identical to the slot 24 provided in the lid 20, as shown in FIGS. 1 to 3. A section of the lid 20 may be configured to be inserted at least partially into the slot provided on the main body 18 to aid in connecting the main body 18 and the lid 20 to each other.

FIG. 5 shows a device 40 for selectively releasing carbon monoxide, including the receptacle 10 shown in FIGS. 1 to 4 and the carbon monoxide releasing main substance 42. As shown in FIG. 5, the main substance 42 may be stored within the chamber 14 of the receptacle 10 and may be configured to emit carbon monoxide such that the emitted carbon monoxide is released through at least a section of the wall 16 of the receptacle 10 after being activated by at least one activation substance 44.

The depiction of the main substance 42 and the activation substance 44 in FIG. 5 is merely exemplary and schematic. The main substance 42 is preferably a powder and/or a granulate. Alternatively, the main substance 42 may be a liquid and/or a gas. Likewise, the activation substance 44 may be a power, a granulate, a liquid and/or a gas.

The activation substance 44 may be introduced into the chamber 14 of the receptacle 10 by means of an introducing device 46. As shown in FIG. 5, the introducing device 46 may be configured as a syringe with a hollow needle 48 configured to pierce through the lid 20, more specifically the protrusion 22, to introduce the activation substance 44 into the chamber 14 to activate the main substance 42. The lid 20 may be configured to reseal, preferably in a substantially liquid-tight and/or gas-tight manner, after the introducing device 46 has been withdrawn from the lid 20.

As discussed above, the receptacle 10, more specifically the device 40, shown in FIGS. 1 to 5 above may be configured for treatment of an animal body and/or a human body in vivo, by releasing the carbon monoxide in vivo. Preferably, the receptacle 10, more specifically the device 40, is configured to be inserted, preferably manually, e.g., by being administered orally, e.g., by swallowing the receptacle 10, more specifically the device 40, and/or rectally and/or into an ear and/or a vagina of the animal and/or a human, into one or more body cavities of a human body and/or an animal body. By doing so, living cells within the human body and/or the animal body may be treated with the carbon monoxide released by the receptacle 10, more specifically the device 40.

However, alternatively, the receptacle 10, more specifically the device 40, may be configured for treatment of biological cells, preferably living cells, ex vivo, by releasing the carbon monoxide ex vivo. Such a treatment system 60 is shown in FIG. 6. The treatment system 60 is configured for treating biological cells 62, preferably living cells, preferably immune cells and/or gametes, with carbon monoxide. The treatment system 60 may be configured to provide one or more effects on the biological cells 62 which at least partially preserve the biological cells 62. The treatment system 60 may include at least one container 64 configured to receive the biological cells 62 and at least one source of carbon monoxide, which in the configuration shown in FIG. 6 is embodied by the device 40 shown in FIG. 5. The container 64 and the source of carbon monoxide device 40 may be arranged relative to each other such that the biological cells 62 may be contacted by the carbon monoxide provided by the device 40 to treat the biological cells 62. The container 64 may include a lid 66, which is preferably configured to provide a seal with the container 64 to seal the contents of the container 64 from an environment, preferably in a gas-tight manner. Alternatively, or additionally, the treatment system 60 may be configured for therapeutically treating an animal body and/or a human body ex vivo, e.g., by applying carbon monoxide to an outer surface, e.g., the skin, of the animal body and/or the human body, which may also be considered as treating biological cells within the meaning of the present disclosure.

FIG. 7 shows, in a schematic view, an alternative treatment system 80 for treating biological cells, preferably living cells, ex vivo, by releasing the carbon monoxide ex vivo. The treatment system 80 may include at least one container 82 configured to receive the biological cells 62 and at least one source of carbon monoxide, which in the configuration shown in FIG. 7 is embodied, as an example but not limited thereto, by the device 40 shown in FIG. 5. The container 82 and the source of carbon monoxide device 40 may be arranged relative to each other such that the biological cells 62 may be contacted by the carbon monoxide provided by the device 40 to treat the biological cells 62. The container 82 may include at least one compartment 84 configured to receive, and preferably house and/or secure, preferably in a captive manner, the device 40. Alternatively, or in addition to the compartment 84, the container 82 may include one or more securing means, e.g., one or more clips, configured to secure, preferably captively and/or substantially immovably, the carbon monoxide releasing device 40 in the container 82. As a further alternative, the device 40 may be omitted and the main substance 42 may be placed directly in at least one chamber defined within the container 82, e.g., directly into the compartment 84. For instance, the compartment 84 itself may form the receptacle 10, e.g., a wall of the compartment 84 may form the housing 12 of the receptacle 10 or at least a portion thereof. This may allow the treatment system 80 to be preassembled, in particular by arranging the device 40 in the compartment 84 prior to use, preferably prior to being distributed to the locations of application, e.g., one or more laboratories. This may facilitate handling and/or use of the treatment system 80.

Preferably, the compartment 84 is arranged at and/or along a bottom of the container 82, in particular such that the biological cells 62 are arranged at least partially above the compartment 84, in particular on an upper wall 86 of the compartment 84, when the treatment system 80 is in an operative state. The upper wall 86 of the compartment 84 may be permeable with respect to the carbon monoxide released by the device 40. This arrangement may increase the exposure of the biological cells 62 to the carbon monoxide released by the device 40. The upper wall 86 may include one or more openings 88 configured to allow the carbon monoxide to pass through the upper wall to the biological cells 62. Alternatively, the compartment 84 may be arranged at and/or along a top and/or at least one side of the container 82.

The container 82 may be configured to receive a plurality of sources of carbon monoxide, e.g., a plurality of devices 40, e.g., by including a plurality of compartments 84, preferably in addition to (and separate from) a compartment configured to receive the biological cells 62, each compartment 84 configured to receive at least one of the sources of carbon monoxide, e.g., of the devices 40.

The container 82 may include a lid 90, which is preferably configured to provide a seal with the container 82 to seal the contents of the container 82 from an environment, preferably in a gas-tight manner. Alternatively, or additionally, the treatment system 80 may be configured for therapeutically treating an animal body and/or a human body ex vivo, e.g., by applying carbon monoxide to an outer surface, e.g., the skin, of the animal body and/or the human body, which may also be considered as treating biological cells within the meaning of the present disclosure.

Any type of triggering mechanism to trigger the release of carbon monoxide from the source of carbon monoxide, e.g., the device 40, in particular when the source of carbon monoxide is arranged in the container 82, more specifically in the compartment 84, and/or the cells 62 are arranged in the container 82, may be provided.

FIG. 8 shows, in a schematic illustration, a further embodiment of the lid 20 of the receptacle 10 shown in FIGS. 1 to 6. The lid 20 shown in FIG. 8 include(s) at least one projection 70 (e.g., rib) which extends from an interface surface 72 of the lid 20. The interface surface 72 may extend along a connection interface between the lid 20 and the main body 18. The projection 70 may extend towards and engage the main body 18, e.g., along an inner surface of the main body 18, when the main body 18 and the lid 20 are connected to each other, e.g., as shown in FIGS. 4 to 6.

The projection 70 may extend at least partially along a circumference, preferably along the entire circumference, preferably multiple times, of the interface surface 72 of the lid 20, preferably in a spiraling manner and/or a thread-like manner, as shown in exemplary manner in FIG. 8, and/or a meandering manner and/or a zig-zag manner. This may provide a relatively large surface along which the projection 70 may provide an increased sealing effect and/or increased friction and/or an increased connection force.

An interface surface of the main body 18, which extends along a connection interface between the lid 20 and the main body 18, is preferably substantially smooth and/or continuous and/or free of projections and/or free of recesses. Alternatively, the main body 18 may have one or more projections and/or one or more recesses, e.g., one or more recesses configured to receive the projection 70.

Alternatively, or additionally, the main body 18 may include at least one projection which is configured similarly or identically to the projection 70.

A distal end 76 of the protrusion 22, which extends furthest into the main body 18 when the lid 20 and the main body 18 are connected to each other, may have one or more edges 94 which are rounded and/or cambered. Alternatively, or additionally, the protrusion 22 may be tapered, at least along a section of the protrusion 22, towards the distal end 76 of the protrusion 22. This may facilitate manufacturing the lid 20, e.g., by enabling an easier removal of the molded lid from a mold compared to a cylindrical shape of the lid 20/protrusion 22 and/or reducing a risk of damage during the removal, if the lid 20 is manufactured by a molding process. In addition, this may prevent, or at least reduce, spillage of a substance, e.g., the activation substance, after removal of an introducing device, e.g., a hollow needle, e.g., the introducing device 46 shown in FIG. 5, for introducing the substance into the receptacle 10, e.g., by leaving a space, or additional space, at the edge(s) of the distal end 76 of the protrusion 22 compared to configuring the protrusion 22 with one or more sharp edges at the distal end 76.

The lid 20 shown in FIG. 8 may have the slot 24 shown in FIGS. 2 and 3.

FIG. 9A shows a correlation between a released amount/volume (in mL) of carbon monoxide (CO) from the receptacle 10 and a mass (in mg) of the main substance 42 (“Mo-CORM”), once the activation substance 44 and the main substance 42 have been combined in the chamber 14 of the receptacle 10. Carbon monoxide (CO) release was 1.30 mL, 0.95 mL, 0.44 mL, and 0.23 mL corresponding to 15 mg, 10 mg, 5 mg, and 2.5 mg of the main substance 42 (“Mo-CORM”), respectively.

FIG. 9B shows a correlation between a released amount/volume (in mL) of carbon monoxide (CO) from the receptacle 10 and a volume (in μL) of the activation substance 44 (“FeCl”), once the activation substance 44 and the main substance 42 have been combined in the chamber 14 of the receptacle 10. Carbon monoxide (CO) release ranged from 0.95 mL to 0.34 mL corresponding to 50 μL to 10 μL of the activation substance 44 (“FeCl”), respectively.

### Illustrative Examples of Treatments of Spermatozoa Using Carbon Monoxide

The following examples illustrate the improvement of characteristics of spermatozoa (like oxidation-reduction-potential, DNA fragmentation and/or spermatozoa motility, in particular progressive motility) when contacted with carbon monoxide gas.

In particular, Example 1 relates to a treatment of spermatozoa using carbon monoxide which is emitted from a treatment system which includes a capsule which has two compartments, as described below in more detail. The results of example 1 are shown in FIGS. 10 and 11.

Example 2 relates to a treatment of spermatozoa using carbon monoxide which is emitted by a treatment system which includes a receptacle, e.g., a capsule, which has a single chamber or compartment defined in a housing, e.g., configured similarly or identically to the receptacle according to any of the embodiments described herein with respect to the first aspect of the present disclosure. The results of example 2 are shown in FIG. 12.

The following results of both examples show that the characteristics of spermatozoa (like oxidation-reduction-potential, DNA fragmentation and/or spermatozoa motility, in particular progressive motility) can be improved when contacted with carbon monoxide gas for various treatment systems, in this case for the different treatment systems used in Examples 1 and 2.

### EXAMPLE 1

### Methods

In this example, the capsule (18-22 mm in length and 6-8 mm diameter) loaded with 15 mg of CO-releasing molecules (Mo-CORM) and 150 μl of an aqueous FeCl3 solution (583.3 mg/ml) was used for the preservation of a 0.7 mL to about 2.9 mL sample in a standard 15 mL Falcon tube.

The capsule, CORM and FeCl3 were prepared as described below:

### Preparation of the Capsule

The capsule was produced by additive manufacturing. The two compartments separated by a septum contained in one of the compartments were 3D printed. The compartment containing the aqueous solution of FeCl3 was printed drop-on-demand (poly-jet modelling process) using an Objet Eden 350 printer (with 96 nozzles, drop size 40 μM, resolution X=600 dpi, Y=600 dpi; Stratasys, Deden Prairie MN, USA). As resin, the photopolymer VeroBlackPlus (Stratasys, Deden Prairie, MN) was used, and the structure was cured via UV-polymerization at 365 nm using a UV lamp. Furthermore, a water-soluble support structure (SUP705, Stratasys, Deden Prairie, MN) was used, which was removed after the printing process by washing steps (potable water; isopropanol).

The compartment containing CORM was produced by laser sintering (LS) using polyamide PA2200 (EOS GmbH, Krailling, Germany) as material, processed by a Formiga P110 3D printer (EOS GmbH, Krailling, Germany), including a CO2-laser (wavelength 10.6 μm, layer thickness 0.1 mm). The silicone membrane (SIK8649), which surrounds the two compartments, was obtained from RAUMEDIC AG (Helmbrechts, Germany). A CAD software (VISI 2019 & 2020, Vero UK, Cheltenham, UK) was used for the production-ready design.

### Preparation of CORM

The synthesis of the CORM trisodiumtricarbonyl-[tris(isocyanoethylacetate)]molybdenum (Na3Mo(CO)3(CNCH2CO2)3, Mo-CORM) was modified from previous protocols (Achatz, D et al. Zeitschrift für anorganische und allgemeine Chemie 2005, 631 (12), 2339-2346). In brief, under dry and inert conditions, 2.64 g (10.0 mmol) of molybdenum hexacarbonyl was dissolved in 35 mL anhydrous acetonitrile (99.8%; 90° C.; 22 h) to yield the intermediate acetonitrile complex. After that, 4.7 mL ethylisocyanoacetate (3.5 eq.; 35 mmol; 4.86 g) was added to replace the acetonitrile at 55° C. to obtain complex. Next, the ester was hydrolyzed using NaOH (16 eq.) in 20 mL tetrahydrofuran at room temperature. By adding aqueous HCl, the free acid is formed. Finally, ethanolic NaOH (5 mmol NaOH/1 mmol Mo-CORM) was added to obtain the trisodium-salt. All reagents were purchased from Sigma Aldrich (Schnelldorf, Germany) and used without further purification.

### Preparation of Aqueous FeCl3 Solution

FeCl3*6H2O was purchased from Sigma Aldrich (Schnelldorf, Germany). An aqueous solution was prepared by preparing a concentration of 583.3 mg/mL with deionized water.

### Sperm Analysis

Between 1.46 mL and 5.7 mL human semen/spermatozoa were collected from healthy males of between 25 and 50 years of age in 100 mL sample vials on site. Samples were then divided into two equal fractions each for subsequent incubation with CO (wCO) and without CO (woCO). In order to estimate the spermatozoa concentration in the samples and to establish an initial baseline for each sample, a routine spermiogram was performed before samples were transferred to gas-tight 15 mL Falcon tubes. Samples were incubated at 25-35° C. for 90 minutes with and without an activated capsule system in a closed container. During this incubation period liquefaction took place naturally. The samples were then analyzed for total motility, progressive motility, static oxidation-reduction potential, and for DNA fragmentation.

Total motility, progressive motility and concentration of the samples were assessed using a CEROS II (Hamilton Thorne, Beverly, MA) computer assisted sperm analysis (CASA) system. 6 μL of a sperm sample diluted 1:1 with Multipurpose Handling Medium-Complete (MHM-C, FujiFilm/IrvineScientific, Santa Ana, CA) was measured and the concentration and level of mobility (total and progressive motility) was assessed.

A Male Infertility Oxidative System (MiOXSYS, Englewood, CO) was used to determine static oxidation-reduction potential (sORP) levels. 30 μL of the sperm samples were transferred to the sample application port on a MiOXSYS sensor and analyzed for 2 minutes.

To determine DNA fragmentation a count-based microscopy assay was performed. For this spermatozoa samples were diluted using MHM-C (Multipurpose Handling Medium-Complete, FujiFilm, IrvineScientific) to a maximum of 20×E6 spermatozoa per mL and analyzed using a Halo Sperm G2 Kit (Halotech, Madrid, Spain). Samples were mixed with the prepared agarose according to manufacturer instructions. 8 μL of the resulting mixture was then put on microscopy slides and cooled down for 5 minutes by removing the heat source. The prepared samples were further treated according to the manufacturer's instructions: denaturant for 7 minutes, lysis solution for 20 minutes, distilled water for 5 minutes, 70% ethanol for 2 minutes, 100% ethanol for 2 minutes, eosin staining solution for 7-10 minutes, and thiazine staining solution for 7-10 minutes. At least 300 spermatozoa were then analyzed using a bright field microscope.

### Results

1. (Static) Oxidation-Reduction Potential (sORP) Levels

“Relative oxidation-reduction-potential” data points in FIG. 10 are derived from the following fraction per test subject: sORP wCOSubject n/sORP woCOSubject n, wherein sORP woCOSubject n was set to 1. sORP data as a measure for oxidative stress (caused among others by ROS) of the sperms/spermatozoa show a trend to be lower in the samples that were contacted with CO (wCO) as compared to samples that were not contacted with CO (woCO). This is evident from the location of the data points, four of which are located below the dashed woCO=1 reference line. Therefore, without being bound by theory, these results indicate that the contacting with CO leads to lower levels of oxidative oxygen species (ROS) (FIG. 10) in spermatozoa as compared to spermatozoa that were not contacted with CO.

### 2. DNA Fragmentation

“Relative DNA fragmentation” data points presented in FIG. 11 are derived from the following fraction per subject: DNA fragmentation wCOSubject n/DNA fragmentation woCOSubject n, whereas DNA fragmentation woCOSubject n was set to 1. As is evident from FIG. 11, DNA fragmentation is reduced in samples that were contacted with CO as compared to samples that were not contacted with CO. This is evident from the location of the data points which are all located below the dashed woCO=1 reference line.

In summary, the results show that the contacting of spermatozoa with CO leads to desired/improved characteristics of gametes (like to a reduction in oxidative stress/oxidation-reduction potential/ROS levels (FIG. 10) and DNA fragmentation (FIG. 11) as well as to an improvement of their progressive motility).

Therefore, carbon monoxide (CO) when contacted to gametes like spermatozoa was surprisingly found by the inventors to lead to preservation of spermatozoa. As a consequence of the co-incubation of gametes with CO, these spermatozoa have been shown to exhibit desired characteristics (compared to spermatozoa that were not contacted with CO) and are thus particularly useful for ART (applications).

### EXAMPLE 2

Example 2 illustrates the improvement of spermatozoan viability parameters (namely, oxidation-reduction potential) in response to different carbon monoxide (CO) incubation durations. Sperm form the same subject was either treated with carbon monoxide or left untreated, as “untreated” control. Treatments with CO varied in duration (60, 90, 180, or 270 min).

### Methods

In this example, sperm samples were treated with either one, three, or six receptacles/capsules, each loaded with 5 mg Mo-CORM (corresponds to 0.44 mL CO) for 60 to 270 mins, or left untreated for the same amount of time.

The receptacle and Mo-CORM were prepared using the materials and as described below. FeCl₃ was used for Mo-CORM activation.

### Materials

All chemicals needed for the synthesis of Mo-CORM as well as activation including molybdenum hexacarbonyl, ethylisocyanoacetate, acetonitrile, anhydrous tetrahydrofuran, sodium hydroxide p.a., hydrochloric acid p.a., absolute ethanol, FeCl₃*6H₂O, nitric acid (65%, p.a.) were purchased from Sigma-Aldrich Chemie GmbH (Schnelldorf, Germany). Multipurpose Handling Medium (MHM) for sperm dilution was purchased from IrvineScientific (Santa Ana, CA). 270 ppm CO calibration gas was purchased from Linde AG (Munich, Germany). The polyamide (PA2200) and duroplast photopolymer MED610+VeroBlackPlus (RGD875) were supplied by EOS GmbH (Krailling, Germany) and Stratasys Ltd. (Rechovot, Israel), respectively. Silicone R 6.65×0.4 mm was purchased from RAUMEDIC AG (Helmbrechts, Germany). SF33 2K-silicone (Mixing ratio 1:1, viscosity before mixing: 7000-8000 cP, stiffness after mixing: 33 ShA, density 1.11 g/cc, breaking point: 4.7 N/mm²) and SF45 2K-silicone (Mixing ratio 1:1, viscosity before mixing: 8500 cP (at 23° C.), stiffness after mixing: 45 ShA, density: 1.12 g/cc, breaking point: 3.5±0.5 N/mm²) were purchased from Silikonfabrik (Ahrensburg, Germany). All casting molds were constructed from polytetrafluorethylene (Vink, Germany) on a CNC machine type neo (Datron AG, Germany). Loctite SI 5248, Loctite 4902, Loctite HY 4011, and Loctite SF7701 were purchased from Henkel (Düsseldorf, Germany). Sterican® needles 0.9*40 mm and Omnican® U100 Insulin needles 0.3*8 mm were purchased from B. Braun (Melsungen, Germany). All other reagents were purchased from Sigma Aldrich Chemie GmbH and at least of pharmaceutical grade unless otherwise noted.

### Manufacturing of the Receptacle

All parts were molded using custom designed Teflon casting molds. After filling the molds, vacuum was applied for 5 minutes to extract any air bubbles. The cast was then filled again to the rim and the vacuum was applied for another 5 minutes. After that time the corresponding cast was pressed into the silicone and fixed with clamps. After drying at 40° C. for 24 hours the casted molds were separated from the cast with pressured air. The housing and lid were casted separately. The chamber was filled with Mo-CORM. The lid and upper rim of the housing were then prepared by applying adhesive primer in some versions. Adhesive was then administered and both parts were pressed together. Moving the two parts against each other in a circular motion leads to an even distribution of the adhesive.

### Synthesis of Mo-CORM

The CORM trisodiumtricarbonyl-[tris(isocyanoethylacetate)]molybdenum (Na3Mo(CO)3(CNCH2CO2)3, Mo-CORM) was synthesized as previously published (Reiländer, ACS Biomaterials Science & Engineering, 2022). In brief, molybdenum hexacarbonyl was stirred in anhydrous acetonitrile to exchange three of its ligands for acetonitrile. These ligands were exchanged for EICA ligands. Purification and cation exchange yielded Mo-CORM as white solid.

### Sperm Preparation

Human sperm was collected from healthy volunteers in 100 mL specimen containers. Afterwards, the ejaculate was split into equal parts and filled into 15 mL gas tight falcon tubes, with one of the samples being exposed to one or more activated receptacles/capsules. Then, both samples were analyzed for static oxidation-reduction potential (sORP) via MiOXSYS after a predetermined time.

### Static Oxidation-Reduction Potential (sORP) Analysis

An oxidative male infertility measuring System (MiOXSYS, Englewood, CO) was used to determine static oxidation-reduction potential (sORP) levels following manufacturer's specifications. 30 μL of sample were transferred to the sample application port onto a MiOXSYS sensor and analyzed for 2 minutes. The results were expressed in milli Volt (mV) and normalized to the seminal sperm concentration (mV/10⁶ sperm/mL).

### Results

FIG. 12 clearly shows the beneficial effects of contacting gametes with carbon monoxide on gamete viability parameters, namely on the oxidation-reduction potential of the treated gametes. This is, inter alia, evident from the gamete samples of subjects 38_7 and 42_1 contacted with carbon monoxide for 60 min and 90 min, respectively. The samples of both subjects show high oxidation-reduction potentials when left untreated (black bars), however a drastic reduction in oxidation-reduction potentials can be observed for the respective samples when treated with carbon monoxide (white bars). Oxidation-reduction potential measurements provide an indication of oxidative stress of gametes, whereas high oxidation-reduction potentials (as in the untreated gamete samples of subjects 38_7 and 42_1, may have negative effects on gamete viability and quality. Accordingly, carbon monoxide (CO) when contacted to gametes like spermatozoa was surprisingly found by the inventors to lead to improved viability parameters and quality of gametes.

1. A receptacle (10) configured to be inserted into a human body and/or an animal body, including:
at least one housing (12);
at least one chamber (14) defined within the housing (12);
wherein the chamber (14) is configured to receive and store at least one main substance (42) configured to selectively emit carbon monoxide, wherein the main substance (42) is configured to emit carbon monoxide after being activated by at least one activation substance (44);
wherein the chamber (14) is enclosed by at least one wall (16) of the housing (12), wherein at least a section of the wall (16) is at least partially permeable to the carbon monoxide emitted by the main substance (42) such that the receptacle (10) can at least partially release the carbon monoxide emitted by the main substance (42) to an environment; and
wherein the receptacle (10) is configured such that the activation substance (44) can be introduced into the chamber (14) from outside of the housing (12), in a state in which the main substance (42) is already arranged within the chamber (14).

2. The receptacle (10) according to claim 1, wherein the receptacle (10) includes only a single chamber (14).

3. The receptacle (10) according to any of the preceding claims, wherein the receptacle (10) is configured to release carbon monoxide in vivo and/or ex vivo.

4. The receptacle (10) according to any of the preceding claims, wherein the receptacle (10) is configured to be administered orally and/or rectally and/or vaginally and/or via an ear into a human body and/or an animal body.

5. The receptacle (10) according to any of the preceding claims, wherein the receptacle (10) is configured to be swallowed by a human and/or an animal.

6. The receptacle (10) according to any of the preceding claims, wherein the housing (12) includes at least one main body (18) and at least one lid (20), wherein the main body (18) and the lid (20) are configured to be connectable to each other, preferably in a substantially liquid-tight manner.

7. The receptacle (10) according to claim 6, wherein the lid (20) and/or the main body (18) include(s) a protrusion (22) which is configured to extend into the chamber (14), when the lid (20) and the main body (18) are connected to each other.

8. The receptacle (10) according to claim 7, wherein the protrusion (22) is configured as a septum which is pierceable by an introducing device (46) configured to introduce the activation substance (44) into the chamber (14) to activate the main substance (42), wherein the protrusion (22) is configured to reseal, preferably in a substantially liquid-tight manner, after the introducing device (46) has been withdrawn from the protrusion (22).

9. The receptacle (10) according to any of claims 6 to 8, wherein the main body (18) and/or the lid (20) include(s) at least one projection (70) which extends from an interface surface (72) of the lid (20) and/or the main body (18), the interface surface (72) extending along a connection interface between the lid (20) and the main body (18), wherein the projection (70) extends towards and engages the other of the lid (20) and the main body (18), when the main body (18) and the lid (20) are connected to each other, preferably wherein the lid (20) or the main body (18) includes the projection (70) and an interface surface of the other of the lid (20) and the main body (18), which extends along the connection interface between the lid (20) and the main body (18), is substantially smooth and/or continuous and/or free of projections and/or free of recesses.

10. The receptacle (10) according to any of the preceding claims, wherein:
the entire receptacle (10) has a size which corresponds to, or is at least not larger than, a standard drug capsule size, preferably a standard drug capsule size from 5 to 000; and/or
the entire receptacle (10) has a size which corresponds to, or is at least not larger than, a standard drug capsule size of 2, 1, or 0.

11. A device (40) for selectively releasing carbon monoxide, including:
the receptacle (10) according to any of the preceding claims; and
at least one main substance (42) configured to selectively emit carbon monoxide, wherein the main substance (42) is stored within the chamber (14) of the receptacle (10) and is configured to emit carbon monoxide such that the emitted carbon monoxide is released through at least a section of the wall (16) of the receptacle (10) after being activated by at least one activation substance (44).

12. A treatment system (60; 80) for treating biological cells (62), preferably living cells, preferably gametes and/or immune cells, with carbon monoxide, including:
at least one container (64; 82) configured to receive the biological cells (62);
at least one source of carbon monoxide (40), preferably at least one device (40) for selectively releasing carbon monoxide according to claim 11;
wherein the container (64; 82) and the source of carbon monoxide (40) are arranged relative to each other such that the biological cells (62) may be contacted by the carbon monoxide provided by the source of carbon monoxide (40) to treat the biological cells (62).

13. A set for treating biological cells (62), preferably living cells, ex-vivo or in-vivo with carbon monoxide, the set including:
at least one device (40) for selectively releasing carbon monoxide according to claim 11; and
at least one activation substance (44), preferably wherein the activation substance (44) is contained within an introducing device (46) with a hollow needle (48) configured to pierce through the device (40) for selectively releasing carbon monoxide, more preferably wherein the hollow needle (48) is configured to pierce through a lid (20) of the device (40) for selectively releasing carbon monoxide.

14. A method of preparing a carbon monoxide delivery device for use, the method comprising:
providing at least one device (40) for selectively releasing carbon monoxide according to claim 11, the device being configured to be inserted into one or more body cavities of a human body and/or animal body, preferably by oral ingestion and/or rectal insertion;
activating the main substance (42) with at least one activation substance (44), by introducing the activation substance (44) into a chamber (14) of the device (40) through a lid (20) or a main body (18) of the device (40).

15. A method of treating a mammal, such as a human, in-vivo with carbon monoxide, the method comprising:
providing at least one device (40) for selectively releasing carbon monoxide according to claim 11;
activating the main substance (42) with at least one activation substance (44), such as by introducing the activation substance (44) into a chamber (14) of the device (40) through a lid (20) or a main body (18) of the device (40);
introducing the at least one device (40) into one or more body cavities of the mammal, such as by oral ingestion and/or rectally.

16. Carbon monoxide for use in treating biological cells, preferably living cells, in and/or outside of a mammal's body, preferably for treating a gastrointestinal disease, wherein the carbon monoxide is released from a device according to claim 11 within the mammal's body and/or outside of the mammal's body.

17. A method of treating biological cells (62), preferably living cells, more preferably ex vivo, with carbon monoxide, the method comprising:
providing a treatment system according to claim 12;
arranging the biological cells (62) in the container (64; 82);
triggering a release of the carbon monoxide from the source of carbon monoxide, wherein the carbon monoxide provided by the source of carbon monoxide contacts the biological cells (62).

18. The method of claim 17, wherein the treatment system is provided by arranging at least one device (40) configured to selectively release carbon monoxide in the container (64; 82), preferably at least one device (40) according to claim 11.

19. The method of claim 17 or 18, wherein triggering the release of carbon monoxide comprises activating a main substance (42) of the source of carbon monoxide with at least one activation substance (44).

20. The method of claim 19, wherein the activation substance (44) is introduced into a chamber (14) in which the main substance (42) is arranged, preferably wherein the activation substance (44) is introduced via an introducing device (46), wherein the introducing device (46) comprises a hollow needle (48) configured to pierce through a wall of the chamber.

21. The method of any of claims 17 to 20, wherein the treatment system comprises at least one first chamber and at least one second chamber, wherein the biological cells (62) are arranged in a first chamber, and wherein at least a main substance (42) of the source of carbon monoxide is arranged in the second chamber, wherein the second chamber is separated from the first chamber by at least one wall (16; 86), wherein at least a section of the wall (16; 86) is permeable to the carbon monoxide emitted by the main substance (42) such that carbon monoxide emitted by the main substance (42) is released from the second chamber into the first chamber.
